(19) United States
(12) Patent Application Publication
(10) Pub. No.: US 2017/0055867 A1
(43) Pub. Date: Mar. 2, 2017

(54) PROTECTION DEVICE, WEARABLE DEVICE, PROTECTION METHOD AND DISPLAY SYSTEM

(71) Applicant: BOE TECHNOLOGY GROUP CO., LTD., Beijing (CN)

(72) Inventor: Hui LI, Beijing (CN)

(21) Appl. No.: 15/159,137

(22) Filed: May 19, 2016

(30) Foreign Application Priority Data
Aug. 31, 2015 (CN) 201510548247.1

(51) Int. Cl. A61B 5/0482; G08B 21/24; A61B 5/00; G01B 11/14; A61B 5/0478; A61B 5/04; G06K 9/00; G01J 1/42

(52) U.S. Cl. CPC A61B 5/0482; G06K 9/00503; G06K 9/00885; G08B 21/24; G01J 1/42; G01B 11/14; A61B 5/0478; A61B 5/04017; A61B 5/742; A61B 5/0006; A61B 5/7455; A61B 5/6803; A61B 2560/0247; A61B 2562/0209; A61B 2503/12

(57) ABSTRACT

A protection device, a wearable device, a protection method and a display system are provided. The protection device of the invention comprises a signal acquisition module, an information processing module, and a reminding module. The signal acquisition module acquires a determination signal, the information processing module processes and analyzes the determination signal acquired by the signal acquisition module and determines whether the user's brain is in a state of fatigue, and the reminding module sends a reminder to the user based on the determination result of the information processing module. The protection device of the invention can monitor the degree of fatigue of a user's brain and determine whether the user's brain is in a state of fatigue, and then send a reminder to the user based on the determination result. The protection device of the invention can be applied to various wearable devices or display systems.

### FIELD OF THE INVENTION

The present invention belongs to the technical field of protection devices, and particularly relates to a protection device, a wearable device, a protection method and a display system.

### BACKGROUND OF THE INVENTION

As electronic display devices such as mobile phones, tablet PCs (Pads) and the like are more and more widely used, cases of typical “student eye diseases” such as myopia, amblyopia, astigmatism and the like are significantly increased. This is because the case that when a user is using one of these electronic display devices, the viewing distance is too short, the viewing environment is not bright enough, or the user is in a state of fatigue is often. However, so far there is no such device that can monitor viewing distance, viewing environment, and state of fatigue of a user.

### SUMMARY OF THE INVENTION

In order to address the problem of visual deterioration resulting from the case that when a user is viewing an electronic display device, the viewing distance is too short, the viewing environment is not bright enough, or the user is in a state of fatigue, the present invention provides a protection device, a wearable device, a protection method and a display system.

As a technical solution adopted to solve the above problem, there is provided a protection device, comprising:

a signal acquisition module for acquiring a determination signal, the determination signal comprising a brain wave from a temporal region of a user;

an information processing module for processing and analyzing the acquired determination signal and determining whether the user's brain is in a state of fatigue; and

a reminding module for sending a reminder to the user based on a determination result of the information processing module.

Optionally, the determination signal comprises a α rhythm, a β rhythm, a θ rhythm and a δ rhythm of the brain wave from the temporal region.

Optionally, the information processing module comprises:

a periodization unit for periodizing the brain wave from the temporal region acquired by the signal acquisition module;

a filtering unit for filtering the periodized brain wave;

an extracting unit for extracting each of the rhythms of the filtered brain wave and calculating an average power spectral density of each rhythm; and

a determination unit for calculating ratios of the average power spectral density of the θ rhythm to the average power spectral densities of the α rhythm, the η rhythm, and the δ rhythm, respectively, and determining whether the user's brain is in a state of fatigue based on the three ratios.

Optionally, the signal acquisition module further comprises:

a distance measurement unit for measuring a distance between the protection device and a display screen; and

a light sensing unit for measuring an ambient light intensity of the protection device,

wherein the determination signal further comprises: the distance between the protection device and the display screen, and the ambient light intensity of the protection device, and

the information processing module further determines whether the distance and the ambient light intensity meet requirements.

Optionally, the distance measurement unit comprises a distance measurement sensor, and the light sensing unit comprises a photosensitive sensor.

Optionally, the brain wave from the temporal region of the user is acquired using a dry electrode.

Optionally, the protection device further comprises:

a communication module for sending the determination signal acquired by the signal acquisition module to the information processing module and sending the determination result of the information processing module to the reminding module.

Optionally, the communication module comprises a Bluetooth module or a wireless network module.

Optionally, the reminding module comprises a buzzer.

The present invention also provides a wearable device, which comprises the above protection device, and is able to fix the protection device to the user's head.

Optionally, the dry electrode comprises an active electrode and a reference electrode, the active electrode is provided at a position on the wearable device near the temporal region of the user, the reference electrode is provided at a position on the wearable device near an underside of an earlobe of the user, the determination signal acquired by the signal acquisition module comprises a potential difference generated between the active electrode and the reference electrode, and the potential difference corresponds to the brain wave from the temporal region of the user.

The present invention also provides a protection method, which employs the above protection device or the above wearable device, and comprises steps of:

acquiring a determination signal by a signal acquisition module, the determination signal comprising a brain wave from a temporal region of a user;

processing and analyzing the determination signal acquired by the signal acquisition module, and determining whether the user's brain is in a state of fatigue, by an information processing module; and

sending, by a reminding module, a reminder to the user based on a determination result of the information processing module.

Optionally, the determination signal comprises a α rhythm, a η rhythm, a θ rhythm and a δ rhythm of the brain wave from the temporal region.

Optionally, the information processing module further performs steps of:

periodizing the brain wave from the temporal region acquired by the signal acquisition module;

filtering the periodized brain wave;

extracting each of the rhythms of the filtered brain wave, and calculating an average power spectral density of each rhythm; and

calculating ratios of the average power spectral density of the θ rhythm to the average power spectral densities of the α rhythm, the η rhythm, and the δ rhythm, respectively, and determining whether the user's brain is in a state of fatigue based on the three ratios.

Optionally, the signal acquisition module further measures: a distance between the protection device and a display screen; and an ambient light intensity of the protection device,

wherein the determination signal further comprises: the distance between the protection device and the display screen, and the ambient light intensity of the protection device, and

the information processing module further determines whether the distance and the ambient light intensity meet requirements.

The present invention further provides a display system, comprising a display device and the above protection device or the above wearable device.

Optionally, the wearable device comprises a hairpin or a hat.

The protection device, the protection method, the wearable device and the display system of the present invention can monitor the degree of fatigue of a user's brain and determine whether the user's brain is in a state of fatigue, and then send a reminder to the user based on the determination result, to remind the user not to view a display screen in a state of fatigue, so as to help regulate the user's unhealthy viewing habit and protect the user's vision. The protection device of the present invention can be applied to various wearable devices or display systems.

### BRIEF DESCRIPTION OF THE DRAWINGS

FIG. 1 is a block diagram of a structure of a protection device of Embodiment 2 of the invention.

FIG. 2 is a block diagram of a structure of the information processing module in FIG. 1.

FIG. 3 is a schematic diagram of a structure of a wearable device of Embodiment 3 of the invention.

FIG. 4 is a schematic diagram of another structure of the wearable device of Embodiment 3 of the invention.

### DETAILED DESCRIPTION OF THE EMBODIMENTS

In order to provide a better understanding of the technical solutions of the present invention to those skilled in the art, the invention is described in further detail below in conjunction with the drawings and specific implementations.

### Embodiment 1

The present embodiment provides a protection device, comprising:

a signal acquisition module for acquiring a determination signal, the determination signal comprising a brain wave from a temporal region of a user;

an information processing module for processing and analyzing the acquired determination signal and determining whether the user's brain is in a state of fatigue; and

a reminding module for sending a reminder to the user based on a determination result of the information processing module.

The protection device of the present embodiment can monitor the degree of fatigue of a user's brain and determine whether the user's brain is in a state of fatigue, and then send a reminder to the user based on the determination result, to remind the user not to view a display screen in a state of fatigue, so as to help regulate the user's unhealthy viewing habit and protect the user's vision. The protection device of the present embodiment can be applied to various wearable devices or display systems.

### Embodiment 2

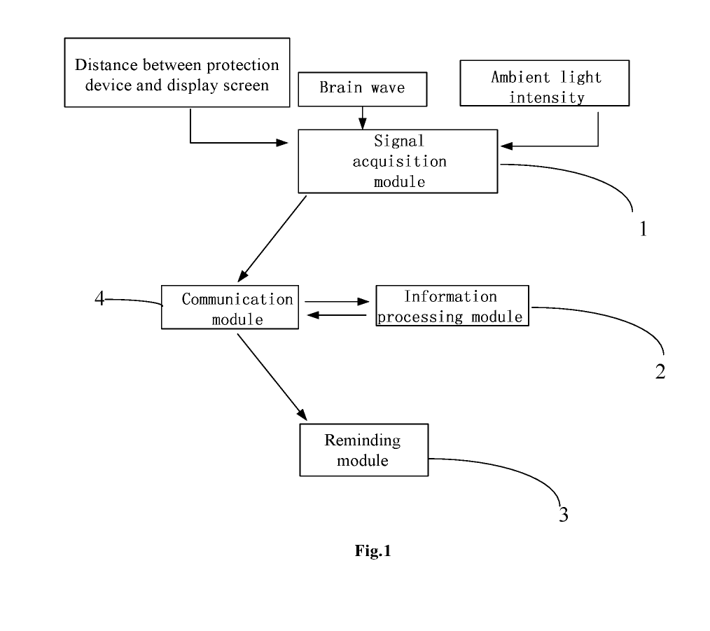

The present embodiment provides a protection device, as shown in FIG. 1, comprising:

a signal acquisition module 1 for acquiring a determination signal, the determination signal comprising a brain wave from a temporal region of a user;

an information processing module 2 for processing and analyzing the acquired determination signal and determining whether the user's brain is in a state of fatigue; and

a reminding module 3 for sending a reminder to the user based on a determination result of the information processing module.

In the present embodiment, first, the signal acquisition module 1 acquires determination signal from the user, then information processing module 2 processes and analyzes the acquired determination signal, to determine whether the user's brain is in a state of fatigue. If the user's brain is in a state of fatigue, then the reminding module 3 sends out a reminder to remind the user to take a break and reduce the fatigue, so as to help regulate the user's unhealthy viewing habit.

Generally, the brain wave from the temporal region includes a α rhythm, a β rhythm, a θ rhythm and a δ rhythm, thus optionally, the determination signal includes the α rhythm, the η rhythm, the θ rhythm and the δ rhythm of the brain wave from the temporal region.

Usually, the α rhythm, the η rhythm, the θ rhythm and the δ rhythm of the brain wave from the temporal region of the user need to be acquired to analyze the degree of fatigue of the user's brain. The θ rhythm with a frequency in the range of 3.75 Hz to 6.75 Hz, which is mainly distributed in the temporal region, may serve as a frequency of normal brain electrical activity of the user, thus, the degree of fatigue of the user's brain may be analyzed mainly based on the θ rhythm.

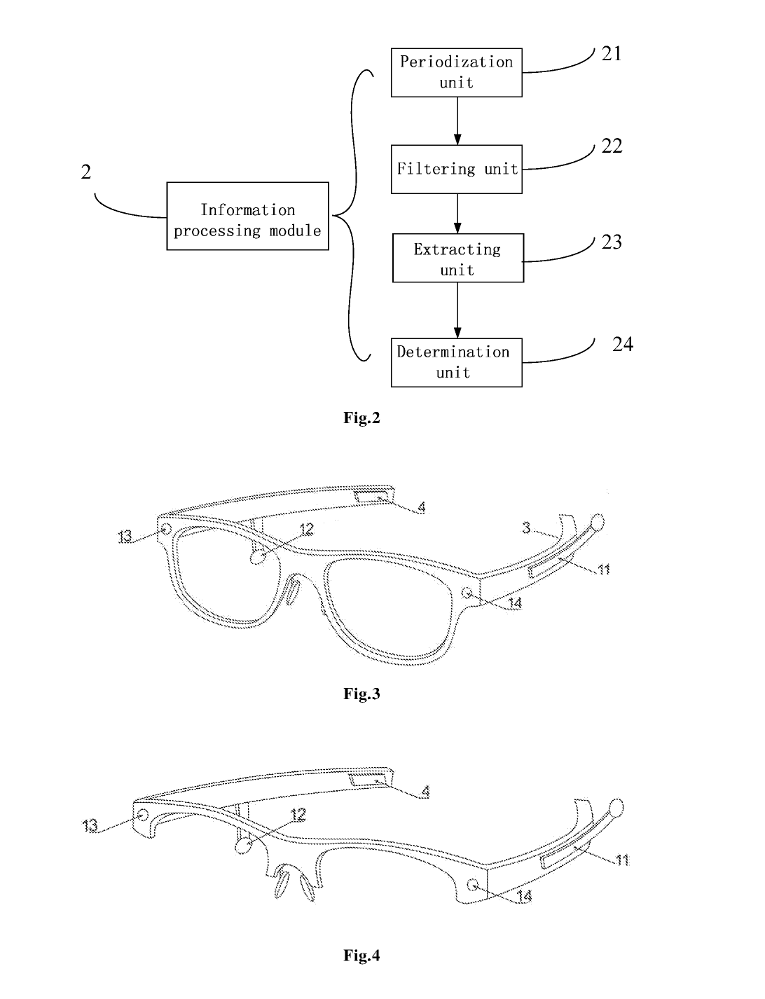

Optionally, as shown in FIG. 2, the information processing module 2 includes:

a periodization unit 21 for periodizing the brain wave from the temporal region acquired by the signal acquisition module 1;

a filtering unit 22 for filtering the periodized brain wave;

an extracting unit 23 for extracting each of the rhythms of the filtered brain wave and calculating an average power spectral density (PSD) of each rhythm; and

a determination unit 24 for calculating ratios of the average power spectral density of the θ rhythm to the average power spectral densities of the α rhythm, the η rhythm, and the δ rhythm, respectively, and determining whether the user's brain is in a state of fatigue based on the three ratios (i.e., the ratio of the average PSD of the θ rhythm to that of the α rhythm, the ratio of the average PSD of the θ rhythm to that of the β rhythm, and the ratio of the average PSD of the θ rhythm to that of the δ rhythm).

Specifically, the brain wave needs to be acquired first; because the acquired brain wave signal has a large amount of data, in order to increase the processing speed, the acquired brain wave need to be periodized; and then the periodized brain wave is filtered, to remove the effect of noises such as myoelectricity or external disturbance; next, each of the rhythms (i.e., the θ rhythm, the α rhythm, the β rhythm, and the δ rhythm) of the brain wave is extracted from the filtered brain wave (for example, by a wavelet decomposition method) and the average PSD of each rhythm is calculated; finally the ratios of the average PSD of the θ rhythm to the average PSDs of all the other rhythms are compared with corresponding predetermined thresholds; if any one of the ratios is lower than the corresponding predetermined threshold, then the user's brain is considered to be in a state of fatigue, and a reminder is sent out by the reminding module 3. The “periodization” in the present embodiment refers to that the brain wave signal in each period of time (e.g., every 30 seconds, every 45 seconds, etc.) is extracted as one signal unit for subsequent processing.

Optionally, as shown in FIG. 3 and FIG. 4, the signal acquisition module 1 includes:

a distance measurement unit 13 for measuring a distance (i.e., a viewing distance) between the protection device and a display screen; and

a light sensing unit 14 for measuring an ambient light intensity of the protection device.

The determination signal further includes: the distance between the protection device and the display screen measured by the distance measurement unit 13, and the ambient light intensity of the protection device measured by the light sensing unit 14, and the information processing module 2 further determines whether the distance and the ambient light intensity meet requirements.

In other words, the signal acquisition module 1 may further acquire the distance between the protection device and the display screen and the ambient light intensity of the protection device at the same time, and the information processing module 2 further determines whether the viewing distance and the ambient light intensity are appropriate based on these acquired data. When the distance between the user and the display screen is too short, or the ambient light intensity is so strong or so weak that it may affect the user's vision, the reminding module 3 sends a reminder to the user, to remind him/her to keep away from the display screen or that the ambient light intensity is too strong or too weak and thus not suited for viewing the display screen.

Optionally, the distance measurement unit 13 includes a distance measurement sensor, and the light sensing unit 14 includes a photosensitive sensor. Preferably, the distance measurement sensor may be an ultrasonic distance measurement sensor or an infrared distance measurement sensor.

Optionally, the brain wave from the temporal region of the user is acquired using a dry electrode.

As shown in FIG. 3 and FIG. 4, the dry electrode includes an active electrode 11 and a reference electrode 12, the determination signal specifically is a potential difference generated between the active electrode 11 and the reference electrode 12, and the potential difference corresponds to the brain wave from the temporal region of the user. Optionally, the active electrode 11 is provided at a temporal region, and the reference electrode 12 is provided on the underside of an earlobe.

Optionally, as shown in FIG. 1, the protection device further includes a communication module 4 for sending the determination signal acquired by the signal acquisition module 1 to the information processing module 2 and sending the determination result of the information processing module 2 to the reminding module 3.

Specifically, when the signal acquisition module 1 and the reminding module 3 are provided on the user's head, and the information processing module 2 is provided in a terminal device such as a computer, the communication module 4 is employed to transmit the determination signal acquired by the signal acquisition module 1 to the information processing module 2, and transmits the determination result of the information processing module 2 to the reminding module 3.

Optionally, the communication module 4 includes a Bluetooth module or a wireless network module.

Specifically, when the signal acquisition module 1 and the reminding module 3 are provided on the user's head, and the information processing module 2 is provided in a terminal device, Bluetooth or wireless network is preferably employed to transmit signals for the user's convenience, and compared to the case where a wired module is employed to transmit signals, entanglement of wires resulting from the movement of the user can be prevented.

Optionally, the reminding module 3 includes a buzzer.

Specifically, when a reminder should be sent to the user according to the determination result of the information processing module 2, the buzzer of the reminding module 3 is enabled and then makes a sound, to remind the user to take a break to relieve fatigue of the brain, or to adjust the viewing distance, or to change the brightness of the surrounding environment, so as to help regulate the user's unhealthy viewing habit.

The protection device of the present embodiment can monitor the degree of fatigue of a user's brain, the viewing distance, and the brightness of the surrounding environment, and determine whether the user's brain is in a state of fatigue, whether the viewing distance is appropriate, and whether the brightness of the surrounding environment is suitable, and then send a reminder to the user based on the determination result, to remind the user not to view a display screen in a state of fatigue, or to adjust the viewing distance, or to change the brightness of the surrounding environment, so as to help regulate the user's unhealthy viewing habit and protect the user's vision. The protection device of the present embodiment can be applied to various wearable devices or display systems.

### Embodiment 3

The present embodiment provides a wearable device, which includes the protection device of Embodiment 2, and can fix the protection device to the user's head.

Optionally, a dry electrode, which includes an active electrode 11 and a reference electrode 12, is employed to acquire the brain wave from a temporal region of the user. The determination signal acquired by the signal acquisition module of the protection device specifically is a potential difference between the active electrode 11 and the reference electrode 12, the potential difference corresponding to the brain wave from the temporal region of the user. The active electrode 11 is provided on the wearable device at a position near the temporal region of the user, and the reference electrode 12 is provided on the wearable device at a position near the underside of an earlobe of the user.

As shown in FIG. 3 and FIG. 4, the wearable device of the present embodiment is shaped like an eyeglass frame. In this case, the distance measurement unit 13 and the light sensing unit 14 are provided at the front frame of the eyeglass frame; the active electrode 11 is provided on the outside of the left leg of the eyeglass frame (or on the outside of the right leg of the eyeglass frame), so that the active electrode 11 is close to the user's temporal region after the user wears the eyeglass frame; the reference electrode 12 is provided on the outside of the right leg of the eyeglass frame (or on the outside of the left leg of the eyeglass frame) and extends downwardly, so that the reference electrode 12 is close to the underside of the earlobe of the user after the user wears the eyeglass frame; the communication module 4 is provided at the inside of the end of the right leg of the eyeglass frame, or at another place on the eyeglass frame; and the reminding module 3 is provided at the inside of the end of the left leg of the eyeglass frame, or at another place on the eyeglass frame.

Apparently, many variations may be made to the specific implementations of the above embodiments, for example, the wearable device may be any device that can be conveniently worn on head, such as a hairpin, a hat or the like.

### Embodiment 4

The present embodiment provides a protection method, which employs the protection device of Embodiment 1 or Embodiment 2, or the wearable device of Embodiment 3, and includes the following steps:

acquiring a determination signal by a signal acquisition module, the determination signal comprising a brain wave from a temporal region of a user;

processing and analyzing the determination signal acquired by the signal acquisition module, and determining whether the user's brain is in a state of fatigue, by an information processing module; and

sending, by a reminding module, a reminder to the user based on a determination result of the information processing module.

Optionally, the determination signal comprises a α rhythm, a η rhythm, a θ rhythm and a δ rhythm of the brain wave from the temporal region.

Optionally, the information processing module further performs steps of:

periodizing the brain wave from the temporal region acquired by the signal acquisition module;

filtering the periodized brain wave;

extracting each of the rhythms of the filtered brain wave, and calculating an average power spectral density of each rhythm; and

calculating ratios of the average power spectral density of the θ rhythm to the average power spectral densities of the α rhythm, the η rhythm, and the δ rhythm, respectively, and determining whether the user's brain is in a state of fatigue based on the three ratios.

Optionally, the signal acquisition module further measures: a distance between the protection device and a display screen; and an ambient light intensity of the protection device.

The determination signal further comprises: the distance between the protection device and the display screen, and the ambient light intensity of the protection device, and the information processing module further determines whether the distance and the ambient light intensity meet requirements. The “periodization” in the present embodiment refers to that the brain wave signal in each period of time (e.g. every 30 seconds, every 45 seconds, etc.) is extracted as one signal unit for subsequent processing.

The protection method of the present embodiment can monitor the degree of fatigue of a user's brain, the viewing distance, and the brightness of the surrounding environment, and determine whether the user's brain is in a state of fatigue, whether the viewing distance is appropriate, and whether the brightness of the surrounding environment is suitable, and then send a reminder to the user based on the determination result, to remind the user not to view a display screen in a state of fatigue, or to adjust the viewing distance, or to change the brightness of the surrounding environment, so as to help regulate the user's unhealthy viewing habit and protect the user's vision.

### Embodiment 5

The present embodiment provides a display system, which includes a display device, and the protection device of Embodiment 1 or 2 or the wearable device of Embodiment 3. The display device may be any product or component with a display function, such as a liquid crystal display panel, electronic paper, an OLED panel, a mobile phone, a tablet PC, a TV, a display, a laptop computer, a digital photo frame, a navigator, or the like.

It can be understood that the foregoing implementations are merely exemplary implementations used for describing the principle of the present invention, but the present invention is not limited thereto. For those of ordinary skill in the art, various variations and modifications may be made without departing from the spirit and essence of the present invention, and these variations and modifications shall fall into the protection scope of the present invention.

1. A protection device, comprising:
a signal acquisition module for acquiring a determination signal, the determination signal comprising a brain wave from a temporal region of a user;
an information processing module for processing and analyzing the acquired determination signal and determining whether the user's brain is in a state of fatigue; and
a reminding module for sending a reminder to the user based on a determination result of the information processing module.

2. The protection device according to claim 1, wherein the determination signal comprises a α rhythm, a η rhythm, a θ rhythm and a δ rhythm in the brain wave from the temporal region.

3. The protection device according to claim 2, wherein the information processing module comprises:
a periodization unit for periodizing the brain wave from the temporal region acquired by the signal acquisition module;
a filtering unit for filtering the periodized brain wave;
an extracting unit for extracting each of the rhythms of the filtered brain wave and calculating an average power spectral density of each rhythm; and
a determination unit for calculating rations of the average power spectral density of the θ rhythm to the average power spectral densities of the α rhythm, the η rhythm, and the δ rhythm, respectively, and determining whether the user's brain is in a state of fatigue based on the three ratios.

4. The protection device according to claim 1, wherein the signal acquisition module comprises:
a distance measurement unit for measuring a distance between the protection device and a display screen; and
a light sensing unit for measuring an ambient light intensity of the protection device,
wherein the determination signal further comprises: the distance between the protection device and the display screen, and the ambient light intensity of the protection device, and
the information processing module further determines whether the distance and the ambient light intensity meet requirements.

5. The protection device according to claim 4, wherein the distance measurement unit comprises a distance measurement sensor, and the light sensing unit comprises a photosensitive sensor.

6. The protection device according to claim 1, wherein the brain wave from the temporal region of the user is acquired using a dry electrode.

7. The protection device according to claim 1, further comprising:
a communication module for transmitting the determination signal acquired by the signal acquisition module to the information processing module and transmitting the determination result of the information processing module to the reminding module.

8. The protection device according to claim 7, wherein the communication module comprises a Bluetooth module or a wireless network module.

9. The protection device according to claim 1, wherein the reminding module comprises a buzzer.

10. A wearable device, comprising the protection device according to claim 1, and being able to fix the protection device to the user's head.

11. A wearable device according to claim 10, wherein the determination signal comprises a α rhythm, a η rhythm, a θ rhythm and a δ rhythm in the brain wave from the temporal region, and the information processing module comprises:
a periodization unit for periodizing the brain wave from the temporal region acquired by the signal acquisition module;
a filtering unit for filtering the periodized brain wave;
an extracting unit for extracting each of the rhythms of the filtered brain wave and calculating an average power spectral density of each rhythm; and
a determination unit for calculating rations of the average power spectral density of the θ rhythm to the average power spectral densities of the α rhythm, the η rhythm, and the δ rhythm, respectively, and determining whether the user's brain is in a state of fatigue based on the three ratios.

12. A wearable device according to claim 10, wherein the signal acquisition module comprises:
a distance measurement unit for measuring a distance between the protection device and a display screen; and
a light sensing unit for measuring an ambient light intensity of the protection device,
wherein the determination signal further comprises: the distance between the protection device and the display screen, and the ambient light intensity of the protection device, and
the information processing module further determines whether the distance and the ambient light intensity meet requirements.

13. A wearable device according to claim 10, wherein the brain wave from the temporal region of the user is acquired using a dry electrode, and the dry electrode comprises an active electrode and a reference electrode, the active electrode is provided at a position on the wearable device near the temporal region of the user, the reference electrode is provided at a position on the wearable device near an underside of an earlobe of the user, the determination signal acquired by the signal acquisition module comprises a potential difference generated between the active electrode and the reference electrode, and the potential difference corresponds to the brain wave from the temporal region of the user.

14. A protection method, comprising steps of:
acquiring a determination signal by a signal acquisition module, the determination signal comprising a brain wave from a temporal region of a user;
processing and analyzing the determination signal acquired by the signal acquisition module, and determining whether the user's brain is in a state of fatigue, by an information processing module; and
sending, by a reminding module, a reminder to the user based on a determination result of the information processing module.

15. The protection method according to claim 14, wherein the determination signal comprises a α rhythm, a η rhythm, a θ rhythm and a δ rhythm in the brain wave from the temporal region.

16. The protection method according to claim 15, wherein the information processing module further performs steps of:
periodizing the brain wave from the temporal region acquired by the signal acquisition module;
filtering the periodized brain wave;
extracting each of the rhythms of the filtered brain wave, and calculating an average power spectral density of each rhythm; and
calculating ratios the average power spectral density of the θ rhythm to the average power spectral densities of the α rhythm, the η rhythm, and the δ rhythm, respectively, and determining whether the user's brain is in a state of fatigue based on the three ratios.

17. The protection method according to claim 14, wherein the signal acquisition module further measures: a distance between the protection device and a display screen; and an ambient light intensity of the protection device,
the determination signal further comprises: the distance between the protection device and the display screen, and the ambient light intensity of the protection device, and
the information processing module further determines whether the distance and the ambient light intensity meet requirements.

18. A display system, comprising: a display device; and the protection device according to claim 1.

19. A display system, comprising: a display device; and the wearable device according to claim 10.
